(19) United States
(12) Patent Application Publication
(10) Pub. No.: US 2026/0345290 A1
(43) Pub. Date: Nov. 5, 2026

(54) Cobotic System for Intraoperative Instrument Sterilization

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/428,205

(22) Filed: Dec. 21, 2025

Related U.S. Application Data
(63) Continuation-in-part of application No. 19/195,731, filed on Apr. 30, 2025.

(51) Int. Cl. A61B 90/70; A61B 34/20; A61B 34/32; A61B 90/98; A61L 2/104; A61L 2/24; A61L 103/15

(52) U.S. Cl. CPC A61B 90/70; A61B 34/20; A61B 34/32; A61B 90/98; A61L 2/104; A61L 2/24; A61L 2103/15; A61L 2202/14

(57) ABSTRACT

A mobile cobotic system for intraoperative instrument sterilization includes a self-contained platform configured for autonomous navigation and positioning within an operating room. The system comprises a robotic manipulation arm for handling surgical instruments, an integrated sterilization module for performing rapid sterilization cycles during surgery, and an instrument identification and tracking system that monitors sterilization status and usage history. A control and monitoring system automates sterilization workflows and verifies completion, while environmental and safety sensors enforce sterile field compliance and collision avoidance. A communication interface enables real-time data sharing with hospital systems, and a user interface provides visual feedback on instrument status and system alerts. The system supports intraoperative instrument reprocessing with minimal disruption to surgical workflow.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to surgical robotics and sterilization systems, and more particularly to a mobile cobotic platform configured for intraoperative instrument sterilization and handling within a surgical environment.

### BACKGROUND

Intraoperative efficiency and sterility are critical to surgical outcomes. During procedures, surgical instruments may need to be re-sterilized or quickly cycled back into use. Traditional sterilization workflows, which typically occur in central sterile processing departments before or after procedures, are not well suited for responsive, intraoperative needs. Moreover, existing intraoperative sterilization techniques often require manual intervention, additional staff, and break the natural surgical rhythm, introducing delays, increasing contamination risk, or diverting clinical attention.

While some portable sterilization units have been introduced for use in or near operating rooms, these systems are generally stationary and lack the ability to autonomously integrate with dynamic surgical workflows. They typically depend on staff to manually place and retrieve instruments and to monitor sterilization parameters and status. Such approaches not only increase the burden on surgical personnel but also introduce variability and increase the likelihood of human error, particularly in high-pressure operative settings.

There is also limited visibility into real-time sterilization status and instrument availability during surgery. Without precise tracking and integration with surgical checklists or procedural timelines, it becomes difficult to know which instruments are clean, which are in use, and which are undergoing sterilization, leading to potential delays or uncertainty at critical moments.

Moreover, the intraoperative environment presents unique challenges with respect to sterility compliance and safety. Any system operating within proximity to a sterile field must maintain strict positional and contamination safeguards. Traditional robotics and automation platforms are often not configured for such nuanced requirements, and most lack integrated sensing or autonomy for safe navigation within an operating room.

Accordingly, there is a need for a mobile, autonomous cobotic system that can sterilize instruments on demand during surgical procedures, handle and transfer instruments without breaching sterile fields, and provide real-time tracking and communication of instrument status. A platform that integrates robotic manipulation, onboard sterilization, environmental awareness, and surgical workflow coordination would fill a critical gap in modern intraoperative logistics.

### SUMMARY

The present invention provides a cobotic system for intraoperative instrument sterilization, designed to operate autonomously within a surgical environment and support real-time surgical instrument handling and reprocessing. The system includes a self-contained mobile cobotic platform equipped with an integrated sterilization module capable of performing rapid sterilization cycles using methods such as ultraviolet-C radiation, steam, plasma, or chemical sterilization.

A robotic manipulation arm mounted on the platform is configured to retrieve surgical instruments, place them into the sterilization module, and remove them after sterilization without breaching sterile field boundaries or interrupting surgical workflow. The system further includes an instrument identification and tracking module that uses radio-frequency identification (RFID), barcode, or QR code recognition to log and monitor the sterilization status, usage history, and availability of each instrument in real time.

An onboard control and monitoring system automates sterilization workflows, from cycle initiation and parameter monitoring to completion verification and alert generation. Environmental and safety sensors detect proximity to sterile fields and potential contamination risks, allowing the platform to autonomously position itself within the operating room while maintaining sterility compliance. The system also includes a user interface that provides visible sterilization status indicators, and a communication interface that transmits data to surgical teams and hospital systems to support intraoperative coordination and inventory management.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of the mobile cobotic sterilization system.

FIG. 2 is a system block diagram of the mobile cobotic sterilization system.

FIG. 3 is a top-down schematic showing the cobot's positioning relative to a sterile field.

FIG. 4 is a flowchart illustrating the sterilization control workflow.

FIG. 5 is a flow diagram illustrating the use of sensor data to enforce sterile field compliance.

FIG. 6 is a diagram depicting communication between the cobotic system and external hospital systems.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

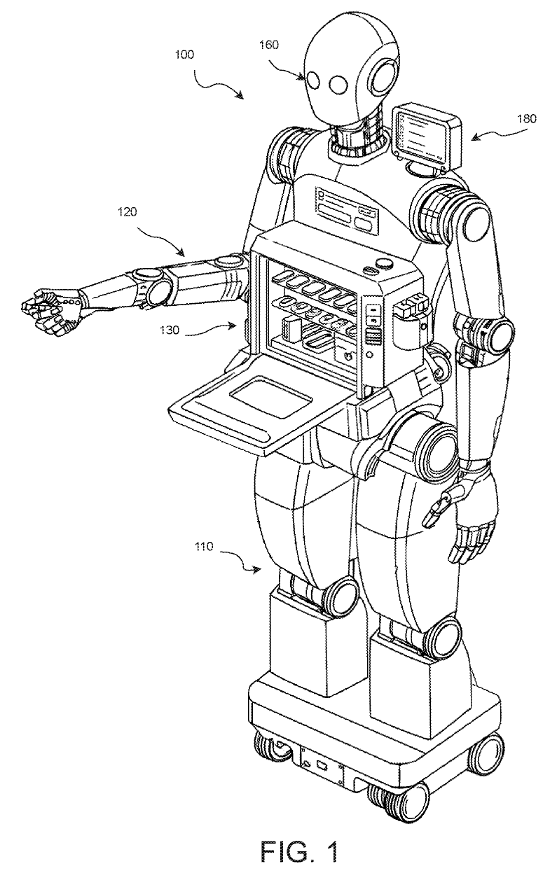

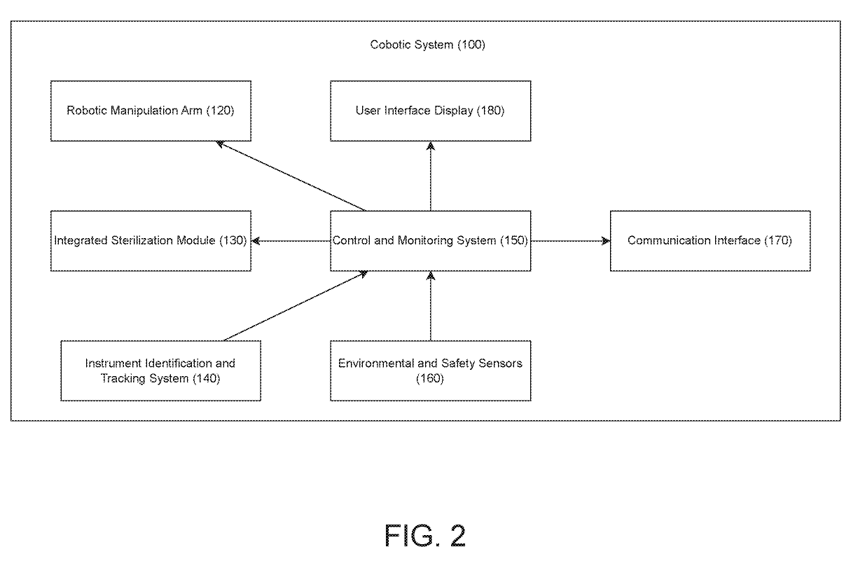

Referring now to the drawings, wherein like reference numerals are used to designate like elements throughout the several views, FIGS. 1 and 2 illustrate a cobotic system 100 for intraoperative instrument sterilization, illustrating an exemplary embodiment of the present invention. Cobotic system 100 is a mobile, self-contained platform that includes a mobile cobotic base 110, a multi-jointed robotic manipulation arm 120, an integrated sterilization module 130, an instrument identification and tracking system 140, a control and monitoring system 150, environmental and safety sensors 160, a communication interface 170, and a user interface display 180. The platform is designed as a collaborative robot (“cobot”) suitable for use in an operating room, with all critical components on-board to enable untethered operation. In the illustrated embodiment, the base 110 supports and houses many of the other components, providing a stable locomotion platform and containing power and computing resources for system 100. The various elements of system 100 are arranged such that surgical instruments can be received, sterilized, and returned in real-time during a surgical procedure without leaving the sterile environment.

The mobile cobotic base 110 is a wheeled or powered cart-like structure that provides mobility and stability for system 100. Base 110 may include omnidirectional wheels or casters driven by electric motors, allowing the system to autonomously navigate the operating room floor. The base houses a power source (e.g., rechargeable battery pack) and the primary electronics, making the unit self-contained and eliminating the need for constant external power or connections during use. In the embodiment of FIG. 1, base 110 includes an upper platform or frame on which the robotic arm 120 and sterilization module 130 are mounted. The base 110 may further incorporate docking receptors or charging contacts (not shown) to recharge the system between uses. The user interface display 180 is also mounted on base 110 in this example, positioned for easy viewing and interaction by operating room staff. The display 180 can be a touchscreen or panel that presents system status (e.g., sterilization progress, instrument ID, alerts) and allows user input or override commands as needed. The mobile base 110 is constructed with a low center of gravity and is configured to remain stationary during critical operations (e.g., when the arm is extended), optionally including brakes or stabilizers for enhanced stability. The combination of an on-board power supply, drive mechanism, and control hardware within base 110 enables system 100 to function as a self-contained cobotic unit that can be easily moved to desired locations in the operating room.

Extending from the base is the robotic manipulation arm 120, which is a multi-jointed robotic arm capable of precise and safe movement for handling surgical instruments. In the illustrated embodiment, arm 120 is mounted to the top of base 110 and includes multiple joints (for example, six degrees of freedom) allowing a wide range of motion. The arm 120 terminates in an end effector or gripper designed to grasp and release surgical instruments securely. This arm is collaborative in nature, meaning it is equipped with force-torque sensors or compliant actuators that allow it to operate safely alongside human personnel. For instance, if the arm 120 encounters an unexpected resistance or contact (such as a human hand or another object), it can automatically slow or stop to prevent injury or damage. In FIG. 1, the arm 120 is shown in a position to transfer an instrument to the sterilization module 130. The reach of arm 120 is sufficient to pick up instruments from standard instrument trays or from a human hand-off position, and to position them into the sterilization module 130 for processing. The arm’s joints are controlled by the system’s control module 150 to perform smooth and accurate movements. In some embodiments, the end effector of arm 120 may be specifically adapted (e.g., with a quick-change tool, gripper fingers, or magnets) to accommodate various types and sizes of surgical instruments, ensuring a secure grasp during transfer. The arm 120 thus provides the core manipulative functionality of the cobotic system, enabling automatic pickup, handoff, and placement of instruments as required during surgery.

Mounted on or within the base is the integrated sterilization module 130, which in this embodiment is a compact sterilizer unit that provides rapid intraoperative sterilization of surgical instruments. As shown in FIG. 1, sterilization module 130 is integrated into system 100 (e.g., built into the side of base 110 or attached as a fixed unit) so that the robotic arm 120 can easily insert and retrieve instruments. In the preferred embodiment, sterilization module 130 is a UV-C based sterilizer chamber: it contains ultraviolet (UV-C) light emitters arranged to irradiate any instrument placed inside, effectively killing or neutralizing microorganisms on the instrument’s surface. The module 130 includes a small closable chamber or drawer, when an instrument is inserted by the arm 120, a door or lid closes to contain the UV-C light and ensure proper exposure. The interior surfaces of the sterilization chamber may be reflective to distribute UV-C light evenly. The sterilization module 130 is configured as a single-mode sterilizer in this example (using UV-C radiation as the primary sterilization mode), which simplifies the design and allows it to be compact and energy-efficient. Environmental sensors 160 (such as UV light sensors or interlock switches) are associated with module 130 to verify that the chamber is closed and sealed before the UV-C lamps activate, thereby preventing any harmful UV exposure to the outside environment. The sterilization cycle time can be predetermined or dynamically adjusted based on the instrument type and contamination level (as assessed by system 140 and 150). In some embodiments, sterilization module 130 may also incorporate heating elements or fans to eliminate any residual moisture or to speed up the process, ensuring the instrument is immediately ready for reuse. Optionally, the sterilization module 130 can be made modular or replaceable; for example, it could be detached for maintenance, or swapped out to employ a different sterilization technique (such as a plasma sterilizer, steam autoclave, or chemical sterilant unit) without replacing the entire cobotic system. In all cases, module 130 is integrated such that system 100 remains a single, mobile unit capable of on-demand instrument decontamination within the operating room.

The cobotic system 100 further comprises an instrument identification and tracking system 140 and various environmental and safety sensors 160, which together enable the robot to intelligently handle instruments and operate safely in a surgical environment. The instrument identification and tracking system 140 includes one or more sensors and associated data processing capabilities that allow the system to recognize and keep track of surgical instruments. In one embodiment, system 140 utilizes a camera or optical sensor mounted near the end effector of arm 120 (or on the sterilization module 130) to capture images of an instrument as it is picked up or before sterilization. The control system 150 then runs image recognition or reads identifying markers (such as QR codes, barcodes, or RFID tags on the instrument) to determine the instrument’s type, size, or ID. This identification informs the control logic about appropriate sterilization parameters (for instance, a required UV-C exposure time or any instrument-specific handling instructions) and also allows the system to log which instruments have been processed. The tracking aspect of system 140 may maintain an inventory status of instruments during the procedure, for example, marking an instrument as “sterilized and ready” after processing, or “used/contaminated” when handed off from the surgical field. In FIG. 1, elements of the identification system 140 (such as a small camera, sensor array, or antenna for RFID detection) are schematically represented on the cobot arm and near the sterilization module. This system 140 is in communication with the control system 150 to provide real-time data on instrument identity and status.

The environmental and safety sensors 160 are distributed on system 100 (for instance, in the eye sockets 105, around base 110, and/or on arm 120) to monitor the surroundings and ensure safe, sterile operation. Sensors 160 may include, without limitation, proximity sensors (e.g., infrared or ultrasonic rangefinders, or LIDAR) to detect nearby staff or obstacles, force-torque sensors in the robot joints to sense contact or resistance, and possibly vision sensors (cameras) that monitor the workspace for any unexpected movement. In the illustrated embodiment, sensors 160 also include dedicated safety interlocks associated with critical components: for example, a sensor to detect that the sterilization module’s door is closed (as noted above), sensors to confirm an instrument is securely gripped by arm 120 before moving, and sensors to detect the boundaries of the sterile field. The data from all sensors 160 feeds into the control and monitoring system 150 so that the robot can dynamically respond, pausing movement if a person comes too close, adjusting arm trajectory to avoid collisions, and ensuring compliance with surgical sterile protocols. Together, instrument identification system 140 and safety sensors 160 imbue system 100 with the awareness needed for autonomous operation in the complex environment of an operating room.

Turning to FIG. 2, a system block diagram of cobotic system 100 is shown, illustrating how the various modules are interconnected and controlled. In this schematic view, the control and monitoring system 150 is depicted as the central controller linking all major components: it is operatively connected to the robotic arm 120, the sterilization module 130, the instrument identification system 140, the sensors 160, the communication interface 170, and the user interface display 180. The control system 150 may comprise one or more processors, microcontrollers or computer modules, along with memory and appropriate interface electronics (motor controllers, sensor interfaces, etc.) for managing the cobot’s operations. In one embodiment, control system 150 is a dedicated onboard computer housed within base 110 that runs control software (including real-time control loops for the robotic arm, motion planning algorithms, image processing for instrument identification, and safety monitoring routines). FIG. 2 shows that control system 150 receives input signals from the sensors 160 and identification system 140, processes this information, and in turn issues command signals to the arm 120 and sterilization module 130. For example, sensor data from a proximity sensor 160 might indicate an obstacle, prompting control system 150 to stop or slow the base 110 or arm 120 movement. Similarly, upon reading instrument identity from system 140, the control system can select an appropriate sterilization cycle in module 130 and command the arm 120 to position the instrument accordingly. The control system 150 is also shown monitoring the status of sterilization module 130 (such as chamber temperature, UV lamp status, or cycle progress) and can control the module’s operation (e.g., turning UV lamps on/off for a defined duration once the instrument is in place). In preferred embodiments, control system 150 executes a safety supervisory routine that, at all times, can override or adjust the robot’s actions if any sensor 160 signals a condition outside safe parameters. The block diagram of FIG. 2 thus underscores that the control and monitoring system 150 acts as the “brain” of cobotic system 100, coordinating all subsystems to work together seamlessly.

FIG. 2 also illustrates the communication interface 170 and user interface display 180 as integral parts of system 100’s connectivity and user interaction. Communication interface 170 is connected to control system 150 and represents the hardware and protocols that allow system 100 to exchange data with external devices or networks. In one embodiment, interface 170 includes a wireless transceiver (for example, Wi-Fi or Bluetooth) enabling the cobot to connect to the hospital’s network or to a dedicated surgical information system. It may also include a wired Ethernet or USB port for direct connections or for maintenance access. Through communication interface 170, real-time data such as instrument IDs, sterilization confirmation, or system status can be sent to external computer systems (e.g., updating a central instrument tracking database or notifying OR personnel via the hospital network). The interface 170 also allows the cobot to receive external commands or configuration updates, for instance, a surgeon’s console could send a request for a specific instrument to be sterilized next, or hospital IT systems could update the sterilization parameters based on the latest protocols. All such communication is handled securely via interface 170, with appropriate encryption or handshaking as needed for patient safety and data integrity.

Meanwhile, the user interface display 180 (also seen in FIG. 1) is connected to control system 150 and provides a local means for human operators to monitor and control the cobot. In the block diagram of FIG. 2, display 180 is depicted as receiving status information from control system 150 (for example, displaying messages like “Instrument identified: Scalpel - Sterilizing now (30s remaining)” or alerts like “Obstacle detected - arm motion paused”). The display 180 can also convey prompts or instructions to the staff, such as indicating when it is ready to receive the next instrument or when a sterilization cycle is complete and an instrument is ready to be retrieved. In some embodiments, user interface 180 is a touchscreen that allows input: an operator could select modes (e.g., switching between automatic and manual operation), acknowledge warnings, or input information (such as confirming the type of instrument if automatic identification is uncertain). The control system 150 is configured to respond to such inputs, thereby incorporating the user’s commands into its operation logic. Additionally or alternatively, physical buttons or emergency stop controls could be present on base 110 (in connection with interface 180) to immediately halt movement or power down in case of an emergency. The block diagram of FIG. 2, therefore, emphasizes that cobotic system 100 is not only autonomous but also network-connected and user-friendly, with interface 170 enabling external data communication and interface 180 facilitating direct human oversight and interaction.

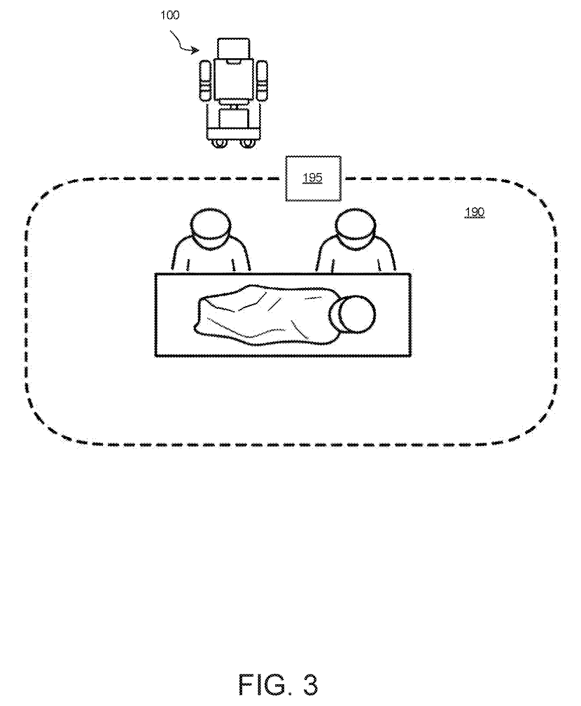

FIG. 3 is a top-down schematic diagram of an operating room deployment of the cobotic system 100, illustrating its proximity to a sterile field (indicated generally at region 190 in the figure) during use. In a typical scenario, sterile field 190 represents the area immediately around the patient and surgical site (often including a Mayo stand or instrument table that holds sterile instruments). System 100 is shown positioned just outside the sterile field boundary, within reach of the surgical staff but far enough to maintain sterility protocols. The mobile base 110 of the cobot can navigate to this position either autonomously or under guidance: for example, prior to surgery, the system may be wheeled into place by staff or drive itself to a designated spot near the operating table. Environmental sensors 160 on board assist in this positioning by detecting walls, the operating table, and other fixed equipment, ensuring that the system stops at a safe distance without bumping into anything. In the top-down view of FIG. 3, the arm 120 is oriented toward the sterile field 190, indicating the area where instruments can be passed between the robot and the human staff. The system’s control logic (via control unit 150) may enforce a virtual boundary corresponding to the sterile field: for instance, the arm 120 will not swing beyond a certain point into the sterile field unless proper conditions are met (such as a sterile drape or interface is present, as described below). This ensures that no non-sterile part of the robot breaches the sterile field inadvertently. The environmental and safety sensors 160 may include vision-based systems or markers that recognize the sterile boundary (for example, a visual marker on the sterile drape or a predefined distance from the patient’s table) so that system 100 can dynamically adjust its working envelope. FIG. 3 thus depicts how the cobotic system is deployed in relation to the surgical sterile area: close enough for convenient handoff of instruments, but with safeguards (both in positioning and sensor feedback) to maintain sterile field compliance and avoid interference with the surgeons or nurses at work.

As shown in FIG. 3, the cobotic system 100 enables an automatic human-cobot handoff of surgical instruments at the edge of the sterile field 190, facilitating seamless collaboration between the robot and operating room staff. In an exemplary use case, when a surgical instrument needs to be sterilized intraoperatively (for example, a tool that has become contaminated or is needed again quickly), a circulating nurse or scrub technician can present or place the instrument at a designated handoff location accessible to the robotic arm 120. This handoff location could be a small sterile tray or platform 195 at the boundary of the sterile field 190, or the nurse might hold the instrument out for the robot to take. The robotic arm 120, guided by its control system 150 and using input from identification sensors 140 and proximity sensors 160, moves to the handoff position in a controlled manner. The arm’s end effector opens and gently closes around the instrument once contact is made, achieving a secure grasp. Because arm 120 is a collaborative robot arm, it can be programmed to move at a slow, deliberate speed during handoff and to use force sensing to confirm that it has the instrument before retracting, this prevents any sudden motions and allows the human operator to let go only when they feel the robot has a firm hold. The system may provide a visual or audible cue (for instance, a light on display 180 or a short beep) to signal that the instrument has been successfully grabbed and the human can release it. Once the instrument is in its grip, arm 120 will retract from the sterile boundary area, ensuring that the instrument (now considered contaminated until sterilized) is moved away from the sterile field 190 into the robot’s domain. Throughout this process, safety sensors 160 monitor the relative positions of the human and any other objects; if, for example, the nurse’s hand is still detected near the instrument, the arm will pause or move extra slowly to avoid any risk of collision. The automatic human-cobot handoff thus allows surgical staff to offload an instrument to the robot with minimal disruption to sterile technique: no one outside the sterile team touches the instrument, and the robot only interfaces at the pre-defined safe zone. After sterilization (discussed further below), a similar handoff in reverse can occur: the arm 120 will extend the now-sterilized instrument back to the edge of sterile field 190, either placing it on a sterile tray or directly handing it to a scrub nurse, again using controlled motions and sensor feedback to ensure a safe exchange. FIG. 3 therefore illustrates how system 100 is employed in the operating room to enable quick, automated instrument reprocessing without violating sterile boundaries and while cooperating closely with human personnel.

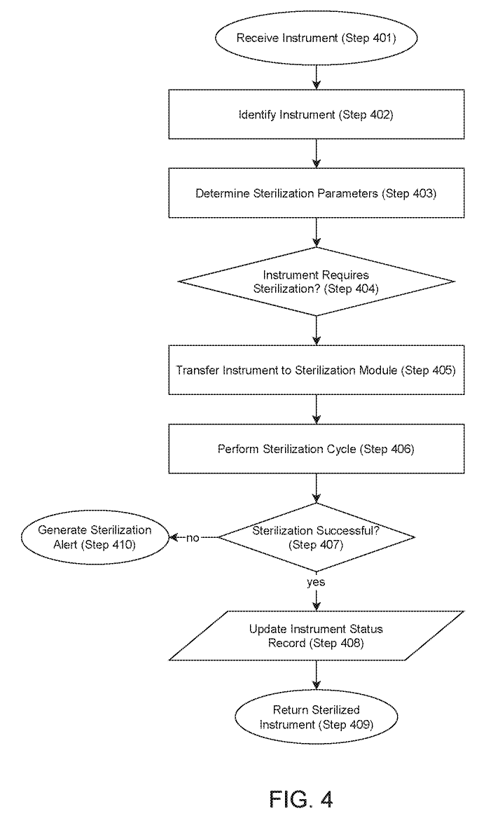

FIG. 4 illustrates a workflow diagram of the instrument identification and sterilization process carried out by the cobotic system during intraoperative operation. The workflow begins at a handoff step in which a contaminated surgical instrument is received by the robotic manipulation arm, such as through a human-cobot handoff at the edge of the sterile field or by retrieval from a designated surface (Step 401). Immediately upon acquisition of the instrument, the instrument identification and tracking system is engaged to determine the specific type or identity of the instrument (Step 402). In one embodiment, this identification step includes capturing image data using a camera or optical sensor to scan the instrument’s shape or identifying markers, and executing image-recognition algorithms or reading machine-readable identifiers such as QR codes, barcodes, or RFID tags associated with the instrument. The control and monitoring system then uses the identification information to determine appropriate sterilization parameters for the instrument, including exposure time, cycle type, or other handling instructions based on the instrument’s size, geometry, material composition, or prior usage history (Step 403). Following parameter determination, the control and monitoring system evaluates whether the instrument requires sterilization prior to reuse during the ongoing surgical procedure (Step 404). While intraoperative use cases will typically require sterilization, this decision step allows the system to verify compatibility with the sterilization module, assess instrument availability, or determine whether alternative instruments are available before proceeding. When sterilization is required, the robotic manipulation arm is commanded to transport the instrument from the handoff location to the integrated sterilization module (Step 405), aligning and positioning the instrument for processing. During this transfer, environmental and safety sensors monitor arm trajectory and placement to ensure proper positioning and compliance with sterile field constraints.

Once the instrument is positioned within the sterilization module, the control and monitoring system initiates and performs the sterilization process using the selected sterilization parameters (Step 406). In one exemplary embodiment, the sterilization module employs ultraviolet-C radiation, activating UV-C emitters for a predetermined or dynamically adjusted duration sufficient to neutralize microorganisms on the instrument surfaces. Throughout the sterilization cycle, environmental and safety sensors verify that the sterilization chamber remains closed and sealed, that required exposure conditions are maintained, and that the platform remains stationary to avoid disturbance during processing. Upon completion of the sterilization cycle, the control and monitoring system evaluates whether sterilization was successfully completed based on sensor feedback, cycle timing, and operational status of the sterilization module (Step 407). If the sterilization cycle completes successfully, the system records the event by updating an instrument status record within the instrument identification and tracking system, marking the instrument as sterilized and ready for reuse and optionally associating a timestamp with the completed cycle (Step 408). The robotic manipulation arm then removes the instrument from the sterilization module and returns the sterilized instrument for intraoperative use, such as by placing it onto a sterile tray, presenting it at a sterile handoff location, or otherwise making it available to surgical staff while maintaining sterility (Step 409). In the event that the sterilization cycle is interrupted, incomplete, or a fault is detected—such as premature chamber opening, sensor failure, or insufficient exposure—the control and monitoring system generates a sterilization alert (Step 410). The alert may be presented via the user interface or transmitted to external systems, prompting human intervention, automatic retry of the sterilization cycle, or other corrective actions before the instrument is returned to use.

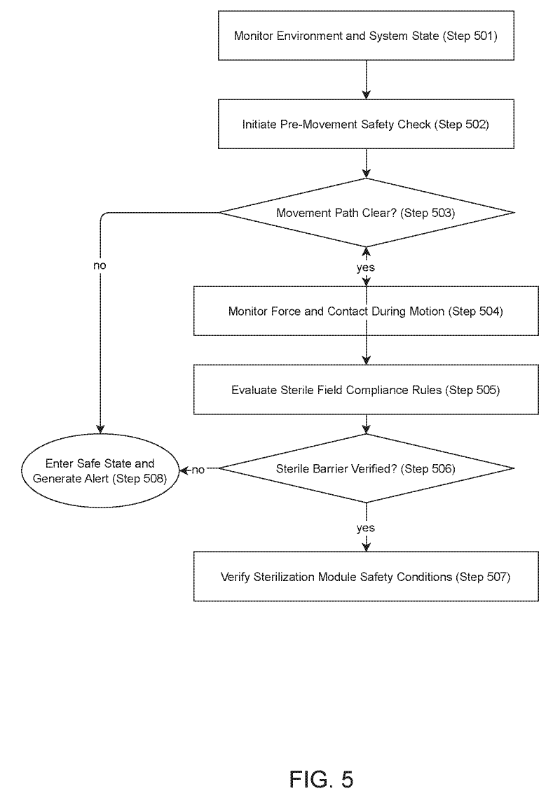

FIG. 5 depicts a sensor-based safety protocol process followed by the cobotic system to maintain sterile field compliance and ensure safe operation at all times. The process illustrated in FIG. 5 continuously monitors the state of the operating environment and the cobotic system, and conditionally allows or restricts system actions based on sensor input (Step 501). In one portion of the process, a pre-movement safety check is performed whenever the robotic manipulation arm or the mobile base is about to move, particularly when movement is directed toward or near the sterile field (Step 502). During this step, the control and monitoring system queries environmental and safety sensors to confirm that an intended movement path is clear of personnel, body parts, or obstacles. For example, before the robotic arm extends to hand off or retrieve an instrument, sensor data from proximity sensors, vision systems, or range-finding sensors is analyzed to determine whether any obstruction is present in the arm’s trajectory. If a surgeon, nurse, or other object is detected within the path, the system delays or halts movement and may generate a warning until the path is clear (Step 503). Similarly, when the mobile base is repositioning, the system verifies that movement will not result in a collision with personnel or equipment; if a potential collision is detected, the base remains stationary or moves at a reduced, controlled speed.

Another portion of the FIG. 5 process involves continuous force and contact monitoring during movement and manipulation (Step 504). Sensor data from force-torque sensors or joint feedback mechanisms is evaluated in real time, and if abnormal resistance, unexpected contact, or collision forces exceeding a threshold are detected, the control and monitoring system immediately halts or back-drives the robotic arm to mitigate potential harm. This logic allows the system to safely respond to unforeseen events, such as accidental contact with personnel or snagging of an instrument, without causing injury or damage.

A further part of the FIG. 5 workflow focuses on sterile field compliance and sterilization safety (Step 505). The control and monitoring system applies predefined rules and sensor inputs to ensure that the sterile field is not compromised by the system’s actions. In one embodiment, when an instrument or robotic component is to cross into or out of the sterile field, the system verifies the presence and integrity of a sterile barrier, such as a sterile drape or sterile transfer tray, using vision-based markers or manual confirmation via the user interface (Step 506). If the sterile barrier is absent or a robotic component is not properly configured for sterile interaction, the system pauses the operation and generates an alert until the condition is corrected.

The safety protocol further includes verification that the sterilization module operates in a safe condition (Step 507). Prior to initiating a sterilization cycle, the system confirms that the sterilization chamber is closed and sealed using door sensors or interlocks, and prevents activation of ultraviolet-C emitters or other sterilization mechanisms if the chamber is not secure. During sterilization, the system may lock the chamber and restrict robotic arm movement to prevent accidental access, and may monitor for conditions such as ambient ultraviolet leakage. If any unsafe condition or system fault is detected—such as sensor failure, chamber opening, or abnormal system status—the control and monitoring system transitions the cobotic system to a safe state (Step 508), halting motion, disabling the sterilization module, and notifying operating room personnel via the user interface or an audible or network-based alert. By adhering to the sensor-driven safety steps illustrated in FIG. 5, the cobotic system maintains sterile field integrity and ensures safe interaction with personnel throughout intraoperative operation.

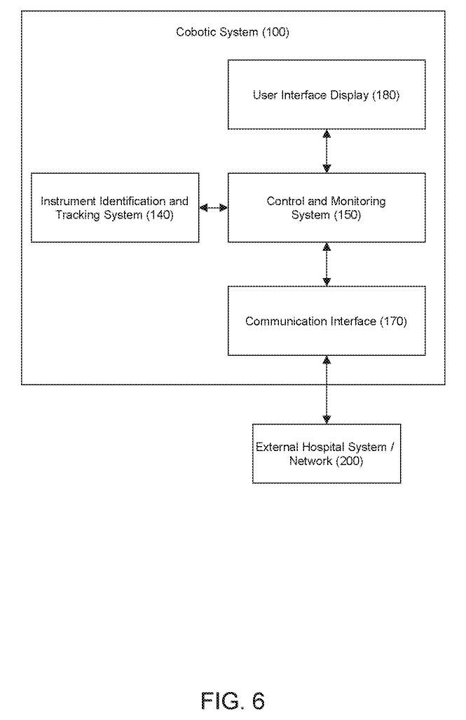

Finally, FIG. 6 illustrates the communication pathways and data flow between the cobotic system 100 and external hospital systems or networks during operation. This figure presents an overview of how information is exchanged to integrate the cobot into the larger surgical and hospital infrastructure. In FIG. 6, the communication interface 170 of system 100 links to a hospital network or central system 200 (for example, a server or database that manages surgical instrument inventory and records). One aspect of the data flow is the instrument tracking data: each time the identification system 140 recognizes an instrument and the control system 150 logs a sterilization event, that information can be transmitted through interface 170 to update the hospital’s instrument management database. For instance, if an instrument has an ID code, system 100 might send a message like “Instrument ID 123 sterilized at 10:43 AM, ready for reuse” to the central system 200. This helps maintain an accurate record of instrument usage and ensures compliance with sterilization documentation requirements. Conversely, the hospital system 200 might provide data to the robot; for example, at the start of a procedure, it could send a list of instruments that are expected to be used or require cleaning, allowing control system 150 to prepare or adjust its settings (such as knowing which instruments to expect and their preferred sterilization cycle parameters). FIG. 6 also shows data flow to and from an operating room control system or surgical scheduling system. The communication interface 170 may receive real-time contextual information, such as the phase of surgery (e.g., “closing incision, no further sterilization needed”), which the control system 150 can use to transition the cobot to a standby or end-of-procedure mode.

Another important communication pathway depicted in FIG. 6 is between the cobotic system 100 and remote monitoring or control interfaces. For instance, hospital personnel or biomedical engineers might have a remote console or tablet (connected via the hospital Wi-Fi through interface 170) that can display the status of system 100 and allow certain controls.

Through such a pathway, a supervisor outside the OR could monitor how many instruments have been sterilized so far, check for any error messages, or even initiate a diagnostic check on system 100 if needed. In some implementations, the cobot can send alerts or maintenance requests over the network, for example, if the sterilization module 130 detects that its UV lamp is nearing end-of-life or if the battery level in base 110 is low, the system might automatically notify the hospital’s engineering department or trigger a service ticket. FIG. 6 may also illustrate communication with external devices in the OR: for example, the cobot could interface with a display in the operating room or with the anesthesia or vital signs monitors to coordinate any required timing (ensuring that sterilization cycles or robot movements do not interfere with critical moments of the surgery). All data transmitted and received via interface 170 is preferably handled through secure protocols, given the medical setting (ensuring patient data, if any, and network integrity are protected). The user interface display 180 is part of this overall communication picture as well, while it is on the robot locally, it serves as a human-machine communication node by showing data that originates from both the robot’s own systems and the hospital systems (for instance, if the central system 200 sends a message like “Procedure extended, additional instrument sterilization may be required,” the control system 150 can display that on interface 180 for the surgical staff to see). In summary, FIG. 6 emphasizes that cobotic system 100 operates not in isolation but as a connected part of the hospital environment, exchanging information with various external systems to enhance efficiency, record-keeping, and safety. Through communication interface 170, the robot becomes an integrated component of the operating theater’s workflow, ensuring that the introduction of this advanced sterilization capability complements existing processes and complies with hospital protocols.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. References herein to “surgical instruments” should be interpreted broadly to include surgical instruments, medical devices used in surgery, and other similar implements that require sterilization in a perioperative environment. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A cobotic system for intraoperative instrument sterilization, comprising:
a self-contained mobile cobotic platform configured for autonomous movement within an operating room and for positioning the system adjacent to a sterile field in compliance with sterile-field protocols;
an integrated sterilization module mounted on the platform and configured to sterilize surgical instruments during a surgical procedure;
a robotic manipulation arm attached to the platform and configured to grasp surgical instruments and transfer the instruments into and out of the sterilization module without compromising sterility or disrupting the surgical workflow;
an instrument identification and tracking system configured to identify individual surgical instruments and monitor a sterilization status of each identified instrument;
a control and monitoring system configured to automate sterilization workflows by controlling the robotic manipulation arm and the sterilization module, and to verify completion of each instrument’s sterilization cycle, including generating status updates or alerts;
one or more environmental and safety sensors configured to detect obstacles and sterile field boundaries, thereby ensuring the platform maintains compliance with sterile field requirements and mitigating contamination risks;
a communication interface configured to share data on instrument status and sterilization progress with external hospital systems in real time; and
a user interface configured to display sterilization progress, instrument status, and alerts to an operator.

2. The system of claim 1, wherein the integrated sterilization module comprises a sterilization chamber that utilizes ultraviolet-C radiation to sterilize the surgical instruments.

3. The system of claim 1, wherein the integrated sterilization module is a modular unit removably attached to the platform, thereby allowing replacement with an alternative sterilization module employing a different sterilization technique selected from steam sterilization, plasma sterilization, or chemical sterilization.

4. The system of claim 1, wherein the robotic manipulation arm comprises an end effector configured to grasp and release surgical instruments, and is further configured to insert an instrument into and remove it from the sterilization module without compromising the sterile condition of the instrument or the surgical field.

5. The system of claim 1, wherein the instrument identification and tracking system comprises at least one of a radio-frequency identification reader or an optical code scanner configured to detect a unique identifier on each surgical instrument.

6. The system of claim 1, wherein the control and monitoring system is configured to automatically initiate a sterilization cycle for an instrument upon detecting that the instrument has been placed into the sterilization module, and to provide an alert indicating when the instrument is sterilized and ready for use or if a sterilization fault is detected during the cycle.

7. The system of claim 1, wherein the environmental and safety sensors comprise one or more sensors selected from optical cameras, infrared sensors, LiDAR sensors, and ultrasonic sensors, the sensors being configured to detect obstacles and sterile field boundaries to prevent the platform from breaching the sterile field.

8. The system of claim 1, wherein the mobile cobotic platform further comprises an autonomous navigation system programmed to navigate within the operating room by using sensor input to avoid obstacles and maintain a safe distance from the sterile field during movement and positioning.

9. The system of claim 1, wherein the communication interface comprises a wireless transceiver configured to transmit sterilization status data and instrument identification information to a central hospital network or database in real time for record-keeping and coordination.

10. The system of claim 1, wherein the user interface comprises a display screen mounted on the platform, the display screen being configured to present real-time sterilization progress of the instruments, the identity of instruments currently being sterilized or awaiting sterilization, and any system alerts to operating room staff.

11. A cobotic system for intraoperative instrument sterilization, comprising:
a mobile cobotic platform configured for autonomous movement and sterile-field-compliant positioning within an operating room;
an integrated sterilization module on the platform for sterilizing surgical instruments;
a robotic manipulation arm mounted on the platform and configured to transfer surgical instruments into and out of the sterilization module;
an instrument identification and tracking system configured to automatically identify each surgical instrument and to monitor the usage and sterilization status of each identified instrument;
a control and monitoring system communicatively coupled to the instrument identification and tracking system and to the robotic manipulation arm, the control and monitoring system being configured to use instrument identification data to automatically control the robotic manipulation arm and the sterilization module for instrument sterilization, and to verify and record the sterilization status of each instrument;
one or more environmental and safety sensors configured to ensure that the platform maintains sterile field compliance and to detect potential contamination risks during operation;
a communication interface configured to transmit data on instrument identity and sterilization status to an external hospital system; and
a user interface configured to display, for each instrument, its identification and current sterilization status, and to provide alerts for instruments requiring sterilization or upon completion of sterilization.

12. The system of claim 11, wherein the instrument identification and tracking system comprises radio-frequency identification (RFID) tags attached to the surgical instruments and an RFID reader on the platform for detecting and identifying each instrument.

13. The system of claim 11, wherein the control and monitoring system includes a data storage medium that maintains a digital log of each instrument’s usage and sterilization history, updating the log every time the instrument is identified and sterilized.

14. The system of claim 11, wherein the control and monitoring system is configured to select a sterilization protocol based on the identified instrument’s type or material, such that different types of instruments automatically receive appropriate sterilization parameters once identified.

15. The system of claim 11, wherein the user interface is configured to display an alert identifying any instrument that requires sterilization during surgery and to indicate when a particular instrument’s sterilization cycle is complete and the instrument is ready for reuse.

16. The system of claim 11, wherein the communication interface is configured to automatically update a central instrument management system with real-time data indicating each instrument’s sterilization status and availability, thereby integrating the cobotic system’s data with hospital inventory or workflow systems.

17. A method for intraoperative instrument sterilization using a mobile cobotic system, the method comprising:
autonomously moving a mobile cobotic platform within an operating room to position an integrated sterilization module proximate to a sterile field;
identifying a surgical instrument in need of sterilization using an instrument identification and tracking system on the platform;
using a robotic manipulation arm on the platform to grasp the identified surgical instrument;
transferring the surgical instrument into the integrated sterilization module on the platform;
sterilizing the surgical instrument within the sterilization module during the surgical procedure;
after sterilization, removing the surgical instrument from the sterilization module using the robotic manipulation arm;
updating the sterilization status of the instrument in a record maintained by a control and monitoring system; and
providing a notification of the instrument’s sterilization status, including indicating completion of sterilization via a user interface or transmitting the status to an external system.

18. The method of claim 17, wherein sterilizing the surgical instrument comprises exposing the instrument to ultraviolet-C radiation within the sterilization module for a predetermined duration to achieve sterilization.

19. The method of claim 17, wherein identifying the surgical instrument comprises using at least one of a radio-frequency identification reader to detect an RFID tag on the instrument, or an optical scanner to read a barcode or QR code on the instrument that is associated with the instrument.

20. The method of claim 17, further comprising scanning the environment using the platform’s environmental and safety sensors to detect obstacles and sterile field boundaries, and automatically adjusting the platform’s path during the moving step to avoid contacting or encroaching upon the sterile field.
